(19) United States
(12) Patent Application Publication
(10) Pub. No.: US 2009/0131893 A1
(43) Pub. Date: May 21, 2009

(54) Fistula connector

(76) Inventors: Peter Andrew Priest, Bristol (GB); Sheila Jean Priest, Bristol (GB)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(21) Appl. No.: 10/585,839

(22) PCT Filed: Jan. 18, 2005
(86) PCT No.: PCT/GB05/00163

§ 371 Date: Jun. 9, 2008

(30) Foreign Application Priority Data
Jan. 19, 2004 (GB) 0400979.1

(51) Int. Cl. A61F 5/448

(52) U.S. Cl. 604/342

(57) ABSTRACT

A connector (1) for connecting a drainage bag (19) around a fistula (23) of a subject, comprising an elongate flexible tubular member (3) having an inlet aperture (5) for receiving waste material from a fistula (23) and an outlet aperture (7) for engagement with a drainage bag (19) through which waste material travels before entering the drainage bag (19). There is also described a drainage appliance incorporating such a connector.

The present invention relates to a connector for forming a connection between a drainage bag or ostomy appliance and a fistula of a subject.

A fistula is an abnormal connection between an organ, vessel or intestine and another organ, vessel or intestine, or the skin. Such an abnormal passage which leaks the contents of the stomach or intestine to the skin is known as an enterocutaneous fistula.

Fistulas are usually the result of trauma or surgery, for example surgery for the treatment of peritonitis, but can also result from infection or inflammation. For example, inflammatory bowel disease, such as ulcerative colitis or Crohn's disease, may lead to fistulas between one loop of intestine and another (enetero-enteral fistula) or intestine and skin (enterocutaneous fistula). One consequence of an operation for the treatment of peritonitis maybe the creation of a fistula through which a portion of the intestine or bowel may protrude.

A stoma is a surgically created opening of the bowel or urinary tract onto the surface of the body. There are usually no nerve endings in a stoma and therefore no sensation.

In the case of an enterocutaneous fistula, the skin surface is often suppurating and the intestine may partially protrude beyond the surface of the skin. The contents of the intestine leak out through the fistula to the skin surface where they must be absorbed using surgical wadding or drained into a drainage bag or ostomy appliance.

The drainage bags or ostomy appliances and means of connection of the bags or appliances to the skin of the user, which are currently used, are designed for patients with an ostomy, that is, a surgically created opening in the body for discharge of body wastes.

Drainage bags or ostomy appliances typically comprise a pouch of plastic, moisture-impermeable and odour-barrier material; an opening in said bag to allow waste material to enter into the plastic pouch; and means to secure the bag in place with the opening connected around the stoma of a patient. This can be achieved by means of a tape that is heat bonded to the ostomy bag around the pouch opening. The tape typically has an opening which is positioned in-line with the opening of the bag and the side of said tape which is adjacent to the patient's body is coated with an adhesive which allows adhesion of the ostomy appliance to the surface of the skin around the opening from which the waste material is leaking.

Often, the means to secure the ostomy bag to the skin surface and the ostomy bag itself, are constructed as two separable parts which are connected and disconnected by the user as desired. This is achieved for instance by applying to the patient's skin, by means of a suitable adhesive or tape that bears on its outer surface, a semi-rigid plastic snap ring, bonded by conventional means to the tape, that surrounds the opening. The ostomy bags then bear a second complementary snap ring mating with the snap-ring on the tape; said complementary snap ring is bonded to the ostomy bag and surrounds the opening therein. The user can therefore apply and remove the ostomy bag without peeling away from the skin the means for securing the bag to the skin surface. When assembled the snap ring provides a tight leak-free seal.

The ostomy bag, which has a surface larger than that of the tape for attachment, lies on the patient's skin, conforming to the abdominal contours and moving with the skin as the skin moves.

However, whilst the use of adhesive to secure the drainage bag or ostomy appliance to the skin of a user is effective for ostomy patients, this means of connection is not appropriate for patients with a fistula since the skin around the fistula is often suppurating and the adhesive tape cannot effectively adhere to such a moist surface.

This has the consequence that the drainage bag or ostomy appliance does not remain in place and is not effectively sealed to the skin around the fistula, thus resulting in leakage of waste material at the surface of the skin of the patient.

This undesirable situation is also exacerbated by movement of the skin surface of the patient such as, for example, bending or stretching whilst moving from a seating to a standing position, and the like.

There is therefore a need for an improved means of sealable connection of a drainage bag around the fistula of a patient.

The present invention seeks to address this problem of the prior art.

Accordingly, a first aspect of the present invention provides a connector for connecting a drainage bag to a fistula of a subject, comprising an elongate flexible tubular member having an inlet aperture for receiving waste material from a stoma and an outlet aperture for engagement with a drainage bag through which waste material travels before entering the drainage bag, the connector being adapted for connection to a fistula entirely by contact with the exterior surface of the subject.

In use, the connector is held against the skin surface of the user thereby providing a means of connecting a drainage bag to the skin surface by means of the connector such that a sealed passage is provided for waste material to exit the fistula and enter the drainage bag.

The flexible nature of the connector allows the connector to contour to the skin surface throughout movement of the patient such as bending and stretching of the abdomen of the patient as occurs in everyday normal activity such as standing up from a chair or bed, and the like.

The connector, once connected to a drainage bag is held in place against the skin of the user by means of a belt or abdominal strap which pushes the connector against the skin surface to retain the sealed passage for drainage of waste materials into the drainage bag, without leakage.

Preferably, the flexible tubular member is resiliently deformable in a longitudinal direction. This allows a greater degree of movement of a drainage bag, once connected to the connector, relative to the point of connection of the connector around the fistula of patient. Thus, for example, a patient bending forwards and stretching backwards would have increased freedom of movement without risk of slippage of the connector and leakage of waste material as the tubular member would be able to be stretched and contracted as necessary. In other words, bending forwards would result in a greater thickness of abdominal tissue around the region of the fistula therefore the tubular member would become stretched longitudinally by pressure from the abdominal tissue thereby preventing the drainage bag, once connected, from digging into the abdomen of the subject.

In order to be resiliently deformable in a longitudinal direction, it is preferably that at least a portion of the tubular member has a ribbed configuration. This allows the tubular member to resiliently deform in a “concertina-like” manner, when required. However, it will be appreciated that other designs allowing resilient deformation in a longitudinal direction are also envisaged. For example, the material from which the tubular member is made maybe capable of resiliently stretching, or a combination of a resiliently deformable material and a ribbed conformation may be used.

Preferably the connector further comprises an inlet flange located at the inlet aperture of the connector. The inlet flange provides an increased surface area for contact with the skin surface of the abdomen around the fistula of the patient. The increased surface area allows for an increased reliability of sealed contact of the connector to the abdominal surface around the fistula, thereby decreasing the risk of detachment of the connector from the abdomen surface and subsequent leakage of fluids upon movement of the abdomen of the patient during normal everyday actions.

Preferably the inlet flange is resiliently deformable. This has the advantage that a greater degree of flexibility of the abdominal surface around the fistula is possible whilst maintaining sealed contact with the inlet flange, which will also flex in a corresponding manner.

Preferably the connector according to the invention further comprises an outlet flange at the outlet aperture of the connector. The presence of a flange at the outlet aperture provides an increased surface area for engagement of the connector to a drainage bag or to an intermediate connector provided for engagement to a drainage bag. As mentioned above, such an increased area for adhesion reduces the likelihood of detachment of the connector from the attached drainage bag or from an intermediate connector for a drainage bag, therefore reducing the likelihood of leakage of fluids from the fistula of the patient at the point of contact of the drainage bag and the connector. The outlet flange may be provided with an adhesive layer on at least a portion thereof for adhesive engagement with a drainage bag or intermediate connector for a drainage bag. The provision of an adhesive layer on the outlet flange has the advantage that separate application of adhesive is not required and therefore the process of attachment of a drainage bag to a connector is simplified and less messy for the patient or nurse. Alternatively, a separate adhesive may be applied prior to connection of the connector to the drainage bag.

The outlet flange may be provided with formations for complementary inter-engagement with corresponding formations on a drainage bag to which it is to be attached. Such formations may take the form of ribs and/or recesses to allow snap-fit inter-engagement of the connector to the drainage bag. Alternatively, the outlet flange may be provided with a screw thread to allow screw-fit inter-engagement with a drainage bag to which it is to be attached.

Preferably, the flexible tubular member of a connector according to the present invention is variable in length. This has the advantage that the flexible tubular member length may be selected dependent on the thickness of the tissue of the abdominal wall around the stoma of the patient. For example, a severely overweight patient may require a connector with a longer flexible tubular member than a patient who is not so overweight, since the depth of abdominal tissue around the fistula of the overweight person is likely to be significantly thicker than the layer of abdominal tissue around the fistula of a less overweight person. Preferably the length of the flexible tubular member may be varied by up to 4 cm in length.

Preferably, the connector is further provided with a retention member to prevent protrusion of an intestine of a patient beyond the connector into the drainage bag. Such a retention member may comprise any suitable partial blockage of the connector sufficient to block movement of the intestine yet still permit waste material to travel therethrough. For example, such a retention member may comprise a grid or mesh-work arrangement, although it will be appreciated that any other suitable structure may be used.

A connector according to the present invention may be formed, at least in part, of a flexible plastics or rubber or foam material.

Preferably, the connector is formed, at least in part, of a porous material adapted to allow passage of gases therethrough, but to prevent substantial egress of liquid. This has the advantage that gasses produced from the material exiting the stoma can exit through the material of the connector, thereby preventing any attached drainage bag from inflating due to produced gases, whilst allowing the selection of the material from which the attached drainage bag is made to be unlimited by the requirement that the drainage bag material be porous to gases and non porous to liquid. For example, it may be preferable that the material of an attached drainage bag is impermeable to gases due to a layer of material which provides greater comfort against the skin of the patient.

Preferably, the material for which the connector is, at least in part, formed, is hypo allergenic in nature.

A potentially suitable material for forming the connector may be Micropore Foam®.

The connector may be manufactured in any suitable way well known to the person skilled in the art. However, preferably the connector is a single piece moulding, thereby reducing the number of join seams present in the connector which may provide a point of weakness through which leakage may occur over time.

A further aspect of the present invention provides a drainage appliance comprising a connector according to a first aspect of the present invention, interconnected with a drainage bag. Any suitable drainage bag known in the art may be used in the manufacture of such a drainage appliance.

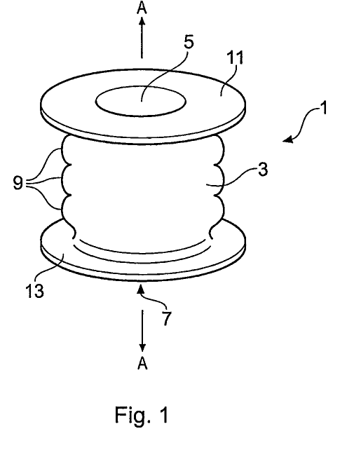

Embodiments of the present invention will now be described, by way of example only, and with reference to FIGS. 1 to 4, in which:

FIG. 1 shows an example of a connector according to the present invention;

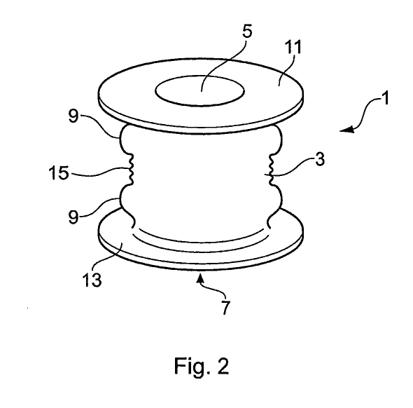

FIG. 2 shows a further example of a connector according to the present invention;

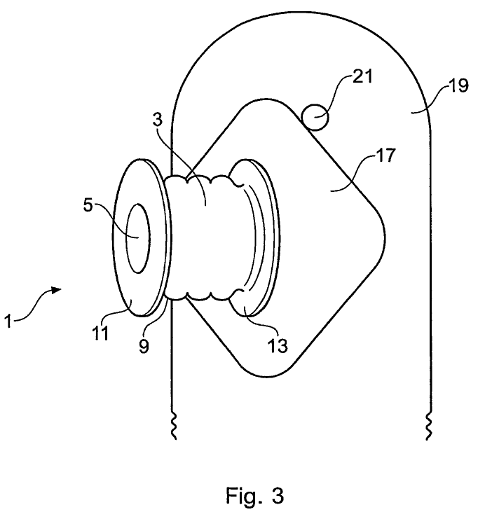

FIG. 3 shows a drainage bag connected to an example of a connector according to the present invention;

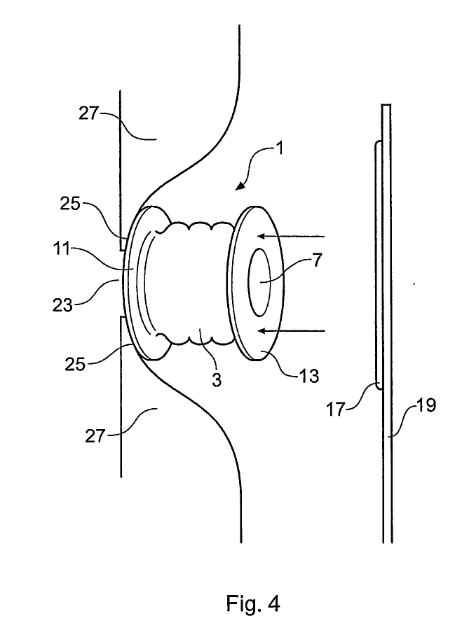

FIG. 4 shows the drainage bag and connector of FIG. 3 in use; and

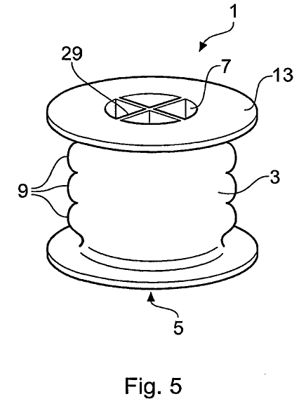

FIG. 5 shows a further example of a connector according to the present invention.

FIG. 1 shows a connector 1 according to the present invention, having an elongate flexible tubular member 3 with an inlet aperture 5 for receiving waste material from a fistula of a patient and an outlet aperture 7 for engagement with a drainage bag (not shown) through which waste material exits the connector and enters the drainage bag. The elongate flexible tubular member is formed with ribs 9 and the inlet aperture 5 is provided with an inlet flange 11, the outlet aperture 7 being provided with an outlet flange 13. In use, the inlet flange 11 is adhered to the abdominal surface of a patient around the fistula of the patient. Waste material exits from the fistula of the patient, enters through the inlet aperture 5 and travels through the flexible tubular member 3 of the connector 1, exiting through the outlet aperture 7 into a drainage bag (not shown) which is in sealable engagement with the outlet flange 13. Once a suitable amount of waste material from the fistula of the patient has traveled through the connector 1 and entered the drainage bag, the drainage bag may be subsequently emptied or replaced. The ribs 9 in the elongate flexible tubular member 3 allow the connector 1 to be resiliently deformable in the longitudinal direction indicated by arrows A.

FIG. 2 shows a further embodiment of a connector according to the present invention, the same reference numerals being used to indicate the same features corresponding to the connector shown in FIG. 1. As well as ribs 9 in the elongate flexible tubular member 3, the connector 1 shown in FIG. 2 is also provided with an extension region 15 which is comprised of a region of finely formed ribs extending annularly around the elongate flexible tubular member 3 and allowing the increase and decrease of the length of the connector 1, by separation of the fine ribs making up the extension region 15, the fine ribs once separated longitudinally from one another in the direction shown in arrows A of FIG. 2, remain in that position until such times as the connector 1 is compressed between the inlet flange 11 and outlet flange 13, thereby pushing the fine ribs of the extension region 15 back together again. In this way, the connector 1 can be adjusted in length so as to be the correct length dependent on the thickness of abdominal tissue present around the fistula of the subject.

FIG. 3 shows the connector 1 of FIG. 1 connected to a connection plate 17 of a drainage bag 19. The outlet flange 13 of the connector 1 is adhered to the attachment plate 17 of the drainage bag 19 by any suitable means, for example, a suitable hypo-allergenic adhesive. The drainage bag 19 is also provided with a filter 21 containing a semi-permeable membrane which allows gas to pass through, but is impermeable to moisture, thereby preventing leakage of any waste fluids entering the drainage bag from the fistula of the patient through the elongate flexible tubular member 3 of the connector 1.

FIG. 4 shows a connector 1 according to the present invention, in place around the fistula 23 of a patient. The inlet flange 11 is in sealed contact with the surface 25 of the abdomen of the patient. The figure reference 27 denotes the abdominal tissue of a patient which rises up and around the connector 1 once adhered in place. It will be appreciated that the thickness of the abdominal tissue determines how long the connector must be to allow non-restrictive movement of the patient without dislodging of the connector from the skin surface and subsequent leakage of waste material. The outlet flange 13 is adhered to the attachment plate 17 of the drainage bag 19 as shown by the arrows in FIG. 4. In other embodiments (not shown) the outlet flange 13 is provided with formations, such as recesses or projections or teeth, for complementary interengagement with corresponding formations on a drainage bag. This may allow, for example, a snap-fit, or a screw fit engagement of the flange with a bag.

Once the connector is in place, the waste material from the fistula 23 of the patient enters through the inlet aperture 5 in the connector 1, travels through the elongate flexible tubular member 3 of the connector 1 and exits the connector 1 through the outlet aperture 7 into the drainage bag 19 via an aperture in the attachment plate 17 of the drainage bag 19. Once a suitable amount of waste material has entered the drainage bag 19, the drainage bag may be either emptied, or removed and replaced with an unused drainage bag.

FIG. 5 shows a connector 1 provided with a retention member 29 located adjacent the outlet flange 13 and spanning the outlet 7. Such a retention member 29 prevents any ingress of an intestine of the subject into a drainage bag connected to the connector 1. Instead, any migrating intestinal portion is prevented from moving past the retention member 29 and cannot progress any further than the connector 1.

1-19. (canceled)

20. Apparatus for connecting a drainage bag to a fistula of a subject, including a connector in the form of an elongate flexible tubular member having an inlet aperture at a proximal end for receiving waste material from the fistula and an outlet aperture at a distal end for engagement with the drainage bag through which waste material travels before entering the drainage bag, and means for applying a force to the connector for pressing the proximal end of the connector into contact with the exterior surface of the subject whereby to connect it to the fistula.

21. Apparatus according to claim 20, wherein the means for applying a force to the distal end of the connector is a belt or abdominal strap.

22. Apparatus according to claim 20, wherein the flexible tubular member is resiliently deformable in a longitudinal direction.

23. Apparatus according to claim 20, wherein at least a portion of the flexible tubular member is ribbed.

24. Apparatus according to claim 20, wherein the apparatus further includes an inlet flange around the inlet aperture.

25. Apparatus according to claim 24, wherein the inlet flange is resiliently deformable.

26. Apparatus according to claim 20, wherein the apparatus further includes an outlet flange around the outlet aperture.

27. Apparatus according to claim 26, wherein the outlet flange is provided with an adhesive layer on at least a portion thereof for adhesive engagement with a drainage bag.

28. Apparatus according to claim 26, wherein the outlet flange is provided with formations for complementary inter-engagement with corresponding formations on a drainage bag.

29. Apparatus according to claim 28, wherein the outlet flange is adapted for snap-fit engagement with a drainage bag.

30. Apparatus according to claim 28, wherein the outlet flange is adapted for screw-fit engagement with a drainage bag.

31. Apparatus according to claim 20, wherein the flexible tubular member is variable in length.

32. Apparatus according to claim 20, wherein the length of the flexible tubular member may be varied by up to 4 cm in length.

33. Apparatus according to claim 20, wherein the apparatus further includes a retention member to prevent movement of an intestine of the subject through the connector into the drainage bag.

34. Apparatus according to claim 20, wherein the connector is formed, at least in part, of a flexible plastics or rubber or foam material.

35. Apparatus according to claim 20, wherein the connector is formed or provided with, at least in part, with an element or member of porous material adapted to allow passage of gases therethrough but prevent substantial egress of liquid.

36. Apparatus according to claim 20, wherein the connector is formed, at least in part, of a hypo-allergenic material.

37. Apparatus according to claim 20, wherein the connector is formed, at least in part, of foam.

38. A drainage appliance comprising apparatus according to claim 20 interconnected with a drainage bag.

39. A method of connecting a drainage bag to a fistula of a subject, using a connector in the form of an elongate flexible tubular member having an inlet aperture at a proximal end for receiving waste material from the fistula and an outlet aperture at a distal end for engagement with the drainage bag through which waste material travels before entering the drainage bag, and means for applying a force to the connector for pressing the proximal end of the connector into contact with the exterior surface of the subject whereby to connect it to the fistula, comprising the steps of placing the inlet aperture of the connector against the fistula and applying the said force to the proximal end of the co
